(19) United States
(12) Patent Application Publication
(10) Pub. No.: US 2019/0185932 A1
(43) Pub. Date: Jun. 20, 2019

(54) METHOD FOR PREPARING LIBRARIES FOR MASSIVELY PARALLEL SEQUENCING BASED ON MOLECULAR BARCODING AND USE OF LIBRARIES PREPARED BY THE METHOD

(71) Applicant: CELEMICS, INC., Seoul (KR)

(72) Inventors: Hyo Ki KIM, Seoul (KR); Hyo Jun HAN, Seoul (KR); Hoon JANG, Seoul (KR)

(21) Appl. No.: 16/304,341

(22) PCT Filed: May 25, 2017
(86) PCT No.: PCT/KR2017/005455

§ 371 Date: Nov. 26, 2018

(30) Foreign Application Priority Data
May 25, 2016 (KR) 10-2016-0063919

(51) Int. Cl. C12Q 1/6874; C12N 15/10; C12Q 1/6855

(52) U.S. Cl. CPC C12Q 1/6874; C12Q 1/6855; C12N 15/1093

(57) ABSTRACT

Provided is a method for preparing libraries for massively parallel sequencing. The method includes: providing two or more double-stranded nucleic acid molecules; ligating adaptors to both ends of each of the nucleic acid molecules; providing a pair of primers for amplifying each nucleic acid molecule wherein each of the paired primers includes i) a 3′-end having a nucleotide sequence complementary to the corresponding adaptor, ii) a 5′-end having a common primer sequence for massively parallel sequencing, and iii) an index sequence located between the 3′-end and the 5′-end, one of the two index sequences is a sequence specific to the corresponding nucleic acid molecule, and the other index sequence is a sequence indexing a sample from which the nucleic acid molecule is derived; and performing amplification using the paired primers to obtain amplification products of the nucleic acid molecules including the molecule-specific sequences and the sample indexing sequences.

### TECHNICAL FIELD

The present invention relates to a method for preparing libraries for massively parallel sequencing based on molecular barcoding and a nucleic acid sequencing method through massively parallel sequencing using libraries prepared by the method.

### BACKGROUND ART

With recent advances in sequencing technologies, next-generation sequencing (NGS) has been well established as an indispensable basic technique in many fields of basic biology, including genomics and transcriptomics. Furthermore, due to various efforts to increase the accuracy of data analysis, NGS has been increasingly utilized in applications (for example, diagnostic applications) where very low error rates are required.

However, despite recent technological developments, the accuracy of NGS is only about 99.9% or less per base and remains far below that of existing sequencing techniques, such as Sanger sequencing. Thus, NGS is currently used in combination with other suitable sequencing techniques, such as Sanger sequencing, in order to avoid the potential risk of misdiagnosis arising from inaccurate sequencing. This combination leads to additional cost and time consumption, which sacrifices the benefits from the introduction of NGS. Under these circumstances, continuous attempts have been made to increase the accuracy of NGS, for example, by statistical and molecular biological approaches. However, most of these attempts should meet several assumptions, require much sequencing data, or incur considerable costs to realize the sequencing technique. Thus, further methodological improvements are needed.

### DETAILED DESCRIPTION OF THE INVENTION

### Problems to be Solved by the Invention

One aspect provides a method for preparing libraries for massively parallel sequencing.

A further aspect provides a nucleic acid sequencing method through massively parallel sequencing using libraries prepared by the method.

Another aspect provides a kit for preparing libraries for massively parallel sequencing.

### Means for Solving the Problems

One aspect provides a method for preparing libraries for massively parallel sequencing, including: providing two or more double-stranded nucleic acid molecules; ligating adaptors to both ends of each of the nucleic acid molecules; providing a pair of primers for amplifying each nucleic acid molecule wherein each of the paired primers includes i) a 3′-end having a nucleotide sequence complementary to the corresponding adaptor, ii) a 5′-end having a common primer sequence for massively parallel sequencing, and iii) an index sequence located between the 3′-end and the 5′-end, one of the two index sequences is a sequence specific to the corresponding nucleic acid molecule, and the other index sequence is a sequence indexing a sample from which the nucleic acid molecule is derived; and performing amplification using the paired primers to obtain amplification products of the nucleic acid molecules including the molecule-specific sequences and the sample indexing sequences.

FIG. 1 is a flowchart showing a method for preparing libraries for massively parallel sequencing according to one embodiment. In step S1, double-stranded nucleic acid molecules are provided as sequencing targets. The double-stranded nucleic acid molecules are naturally occurring or synthetic nucleic acid molecules. Step S1 may include making both ends of each nucleic acid molecule blunt-ended (end repair). Step S1 may include appending one deoxyadenosine base on each of the 3′-ends of the nucleic acid molecules (deoxyadenosine (dA)-tailing) such that adaptors are ligated to both ends of each nucleic acid molecule in a predetermined direction. The deoxyadenosine tailing is usually performed using T4 DNA polymerase and Klenow fragment but is not limited thereto. Step S1 may include phosphorylating both 5′-ends of each nucleic acid molecule (phosphorylation). The phosphorylation can be performed using a suitable enzyme, such as T4 polynucleotide phosphorylase. Step 1 may further include purifying the nucleic acid molecules before and after the end repair and the deoxyadenosine tailing.

The naturally occurring double-stranded nucleic acid molecules may be cell-derived DNA molecules or cell-free DNA molecules. The nucleic acid molecules may be DNA molecules derived from animal cells or body fluids. For example, the nucleic acid molecules may be DNA molecules present in a very small amount in blood such as circulating tumor DNAs or a small amount of damaged DNA molecules such as DNAs derived from formalin-fixed paraffin-embedded (FFPE) tissue. The naturally occurring nucleic acid molecules may be fragmented to a predetermined size before use. This fragmentation can be performed by sonication, heat or enzymatic treatment. Examples of enzymes suitable for the fragmentation include transposases, such as Tn5 transposase and Tn3 transposase, integrases, and recombinases.

In step S2, adaptors are ligated to both ends of each of the nucleic acid molecules. T4 DNA ligase, T7 DNA ligase or a ligase capable of undergoing temperature cycling may be used for ligation of the adaptors. A ligase may also be used that joins double-stranded nucleic acid molecules with higher efficiency than does single-stranded nucleic acid molecules.

The adaptors may be those that are usually used in the preparation of libraries for massively parallel sequencing. Each of the adaptor does not need to include an index sequence to identify a sample or the nucleic acid molecules. Each adaptor may be in the shape of a “Y” or may have a hairpin structure. When the adaptor has a hairpin structure, the method may further include enzymatically cleaving the inner region of the ligated adaptor. For example, an enzyme such as uracil-specific excision reagent (USER) may be used to cleave uracil residues in the adaptors. Due to this enzymatic cleavage, the hairpin-shaped ends of the nucleic acid molecules can be deformed into “Y” shapes.

In step S3, a pair of primers are provided to amplify each nucleic acid molecule. Each of the paired primers includes i) a 3′-end having a nucleotide sequence complementary to the corresponding adaptor, ii) a 5′-end having a common primer sequence for massively parallel sequencing, and iii) an index sequence located between the 3′-end and the 5′-end. When one primer (e.g., a forward primer) of the paired primers includes a sequence specific to the corresponding nucleic acid molecule as the index sequence, the other primer (e.g., a reverse primer) includes a sequence indexing a sample. The index sequences are not in the form of a homopolymer or hairpin. This reduces the possibility of errors in subsequent sequencing.

The molecule-specific sequences are barcode sequences that bind specifically to the nucleic acid molecules to distinguish the nucleic acid molecules from other nucleic acid molecules. The molecule-specific sequences are also referred to as “molecular barcoding sequences” or “molecular indexing barcodes”. The length of each molecule-specific sequence may be controlled taking into consideration the number of the nucleic acid molecules. Each of the molecule-specific sequences may consist of 4 to 20 nucleotides, 4 to 16 nucleotides, 4 to 12 nucleotides, 4 to 10 nucleotides or 6 to 8 nucleotides. The molecule-specific sequences may be randomly synthesized. The expression “randomly synthesized” means that the sequence is not composed of only one base selected from A, G, T, and C bases.

The sample indexing sequences are barcode sequences specifically assigned to a sample before massively parallel sequencing of a mixture of a plurality of samples. The sample indexing sequences function to index a sample from which reads are derived. The sample indexing sequences are also referred to as “sample barcode sequences” or “or sample indexing barcodes”.

In step S4, amplification is performed using the paired primers to obtain amplification products. In each of the amplification products, the molecule-specific sequences and the sample indexing sequences are present in both flanking regions of the nucleic acid molecule.

The amplification may be PCR amplification using the paired primers. The number of reaction cycles for the PCR can be minimized. Thus, the method requires a reduced number of PCR cycles for the introduction of the index sequences, resulting in a decrease in the likelihood of PCR duplicates, compared to conventional methods for the introduction of index sequences by ligation. The number of the amplification cycles may vary depending on the amount of the sample and may be, for example, 16 or less, 14 or less, or 12 or less. The number of the amplification cycles may be in the range of 4 to 16, 4 to 14, 4 to 12, 6 to 16, 6 to 14, or 6 to 12.

FIGS. 2a to 2d are schematic diagrams showing embodiments of a method for preparing libraries for massively parallel sequencing. As shown in FIGS. 2a to 2d, various forms of adaptor molecules can be ligated to nucleic acid molecules. A molecule-specific sequence may be present in one of a pair of primers and a sample indexing sequence may be present in the other primer.

FIG. 3 is a flowchart showing a method for preparing libraries for massively parallel sequencing according to a further embodiment. As shown in FIG. 3, the method may further include capturing amplification products as sequencing targets. The target amplification products are the nucleic acid molecules including target loci. Due to this step, a high sequencing depth for the target loci can be achieved. The capture is also referred to as “target capture” or “target enrichment”.

The capture may be performed by hybridization. In this case, nucleic acid probes capable of complementary binding to the target loci are constructed, the nucleic acid probes are brought into contact with libraries, and only nucleic acid molecules including the target loci are sorted. The hybridization may be solution-based hybridization. Some bases of the probe molecules may be biotinylated. The nucleic acid molecules hybridized with the probes including the biotinylated bases can be selectively separated using streptavidin-coated beads.

The method may further include amplifying the captured products to recover at least a portion of the amount of the nucleic acid sample reduced after the capture step. The captured products can be amplified using the common primer sequences. Since the amplification has no influence on the index sequences present in the captured products, PCR duplicates produced in this step can be removed by subsequent analysis of the index sequences.

A further aspect provides a nucleic acid sequencing method through massively parallel sequencing, including: subjecting the libraries prepared by the method to massively parallel sequencing to obtain reads; removing duplicates having the same molecule-specific sequences and sample indexing sequences as the reads; and sequencing the deduplicated reads.

FIG. 4 is a flowchart showing a nucleic acid sequencing method through massively parallel sequencing according to one embodiment. Steps S1 to S4 are as described above. In step S5, the amplification products are subjected to massively parallel sequencing. The massively parallel sequencing may be carried out by any suitable technique for parallel sequencing of nucleic acid molecules. The massively parallel sequencing is also referred to as next-generation sequencing (NGS)” or “high-throughput sequencing”. The massively parallel sequencing is selected from the group consisting of sequencing by synthesis, Ion Torrent sequencing, pyrosequencing, sequencing by ligation, nanopore sequencing, and single-molecule real-time sequencing, but is not limited thereto.

In step S6, duplicates are removed from the reads obtained by the sequencing. The duplicates refer to reads obtained as a result of re-amplification of the primers annealed to the amplification products during the preparation of libraries for sequencing. The duplicates may change the proportions of the original DNA molecules and the amplified DNA molecules. For example, the duplicates may negatively affect the detection of genetic variation through analysis of the reads. Some reads identified to have the same molecule-specific sequences and sample indexing sequences during sequencing of the reads can be considered duplicates. The duplicates can be removed by an algorithm capable of identifying the index sequences and grouping a plurality of reads according to the index sequences. The algorithm may be any of those known in the art. The algorithm may also be home-made.

In step S7, the deduplicated reads are sequenced. The sequencing may include aligning the deduplicated reads to a reference sequence. The reference sequence may be sequence information stored in a sequence database known in the art. The reads can be aligned using a sequence alignment tool known in the art or a home-made tool for read alignment. Examples of such sequence alignment tools include, but are not limited to, BWA, BarraCUDA, BBMap, BLASTN, Bowtie, NextGENe, and UGENE.

The method may not involve additional steps of removing some of the reads mapped to the same location of the reference sequence during sequencing as duplicates, which is the different duplication removing step mentioned above. Preferably, the method does not further involve removing duplicates after duplicate removal step S6. The method may not involve implementing an algorithm, such as Markduplicates algorithm from Picard, to remove duplicates based on the alignment locations of the reads, with the result that the sequencing depth value increases, ensuring a broad area where the necessary amount of data for analysis can be acquired.

The method may further include comparing the sequences of the aligned reads mapped to the target loci to detect sequence variants. Since the method leads to an overall increase in sequencing depth, as described above, a sufficient amount of data at the target loci can be acquired even after the removal of duplicates, ensuring highly sensitive and accurate detection of sequence variants.

In this step, when the proportion of reads having the same sequence variants in the reads mapped to the target loci is below a predetermined value, the sequence variants can be determined to be caused by sequencing errors. The predetermined value can be determined according to the target sequences or other purposes. For example, the predetermined value may be in the range of 30% to 95%, 40% to 95%, 50% to 90%, 60% to 90%, 70% to 85% or 75% to 80% for germline variants. The predetermined value may vary depending on the kind of an analyte sample. For example, the analyte sample may be a tumor sample. In this case, the predetermined value may be lowered depending on various factors, such as the ratio of normal cells to tumor cells in the sample. When the ratio is above the predetermined value, the sequence variants can be considered to be actually present in the nucleic acid molecules.

FIG. 5 is a flowchart showing a nucleic acid sequencing method through massively parallel sequencing according to a further embodiment. As shown in FIG. 5, the deduplicated reads can be analyzed to detect sequence variants present in the target loci.

Another aspect provides a kit for preparing libraries for massively parallel sequencing, including a plurality of pairs of primers wherein each of the paired primers includes a 3′-end having a nucleotide sequence complementary to adaptors ligated to both ends of a nucleic acid molecule, a 5′-end having a common primer sequence for massively parallel sequencing, and an index sequence located between the 3′-end and the 5′-end, one of the two index sequences is a sequence specific to the nucleic acid molecule, and the other index sequence is a sequence indexing a sample from which the nucleic acid molecule is derived.

The number of the primer pairs in the kit can be controlled depending on the number or amount of the nucleic acid molecules. The kit may further include one or more of the following: adaptor molecules, dNTPs, enzymes, probe reagents, reaction reagents, buffers, beads, reaction containers, storage containers, and experimental protocols. The kit is suitable for use in the method for preparing libraries for massively parallel sequencing.

The molecule-specific sequences and the sample indexing sequences are the same as those described above. The length of the molecule-specific sequences can be controlled taking into consideration the number of the nucleic acid molecules. For example, the molecule-specific sequences may consist of 4 to 20 nucleotides. Products obtained by amplification using the primers may include the molecule-specific sequences and the sample indexing sequences in the flanking regions of the nucleic acid molecules.

### Effects of the Invention

The method for preparing libraries for massively parallel sequencing according to one aspect can be used for efficient sequencing through massively parallel sequencing. Specifically, index sequences can be introduced in a more efficient manner and PCR duplicates can be removed in a more effective manner according to the method than according to conventional methods based on ligation. In addition, the use of libraries prepared by the method ensures more accurate detection of sequence errors or rare sequence variants present in target loci.

### BRIEF DESCRIPTION OF THE DRAWINGS

FIG. 1 is a flowchart showing a method for preparing libraries for massively parallel sequencing according to one embodiment.

FIGS. 2a, 2b, 2c and 2d are schematic diagrams showing embodiments of a method for preparing libraries for massively parallel sequencing.

FIG. 3 is a flowchart showing a method for preparing libraries for massively parallel sequencing according to a further embodiment.

FIG. 4 is a flowchart showing a nucleic acid sequencing method through massively parallel sequencing according to one embodiment.

FIG. 5 is a flowchart showing a nucleic acid sequencing method through massively parallel sequencing according to a further embodiment.

FIGS. 6a and 6b are a flowchart showing a general method for analyzing massively parallel sequencing data and an algorithm used in the method, respectively.

FIGS. 7a and 7b are a flowchart showing a method for analyzing massively parallel sequencing data according to one embodiment and an algorithm used in the method, respectively.

FIGS. 8a, 8b and 8c compare the results of analysis of sequencing data from three samples by a method according to one embodiment and a conventional method.

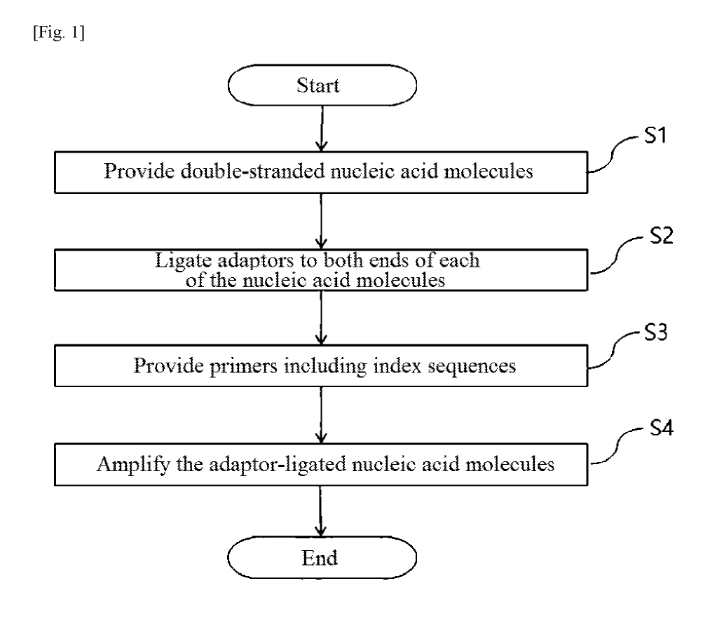

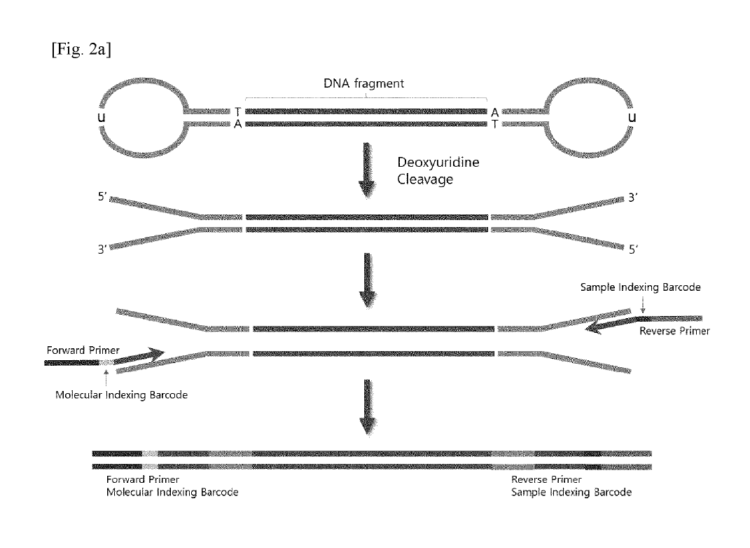

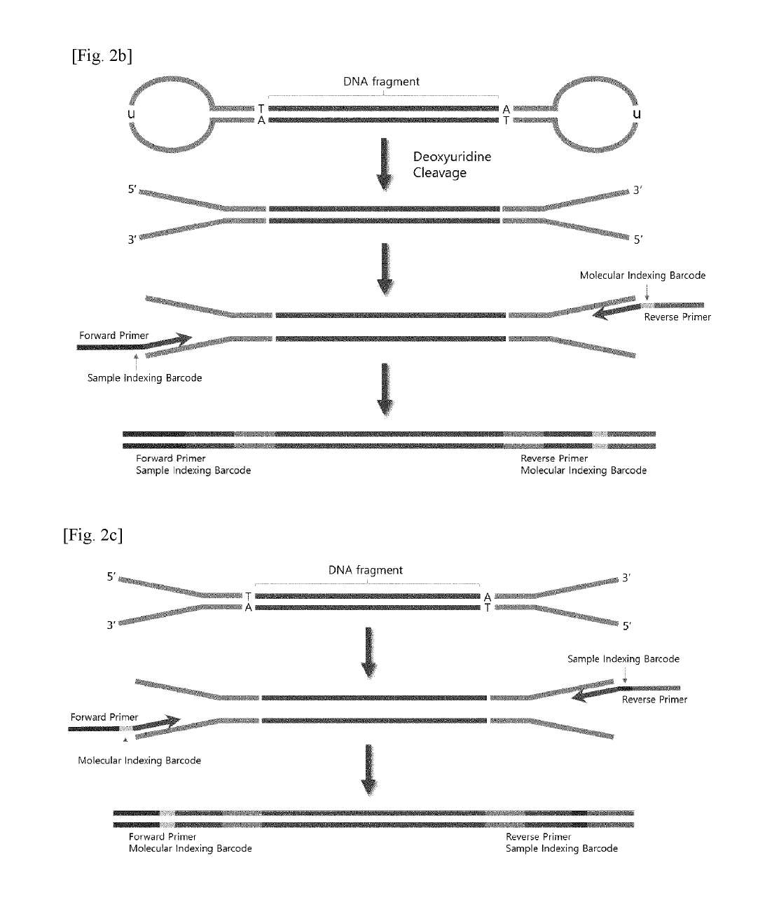

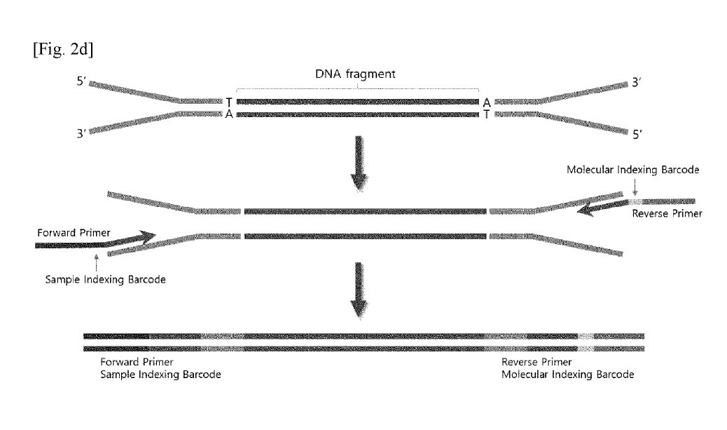

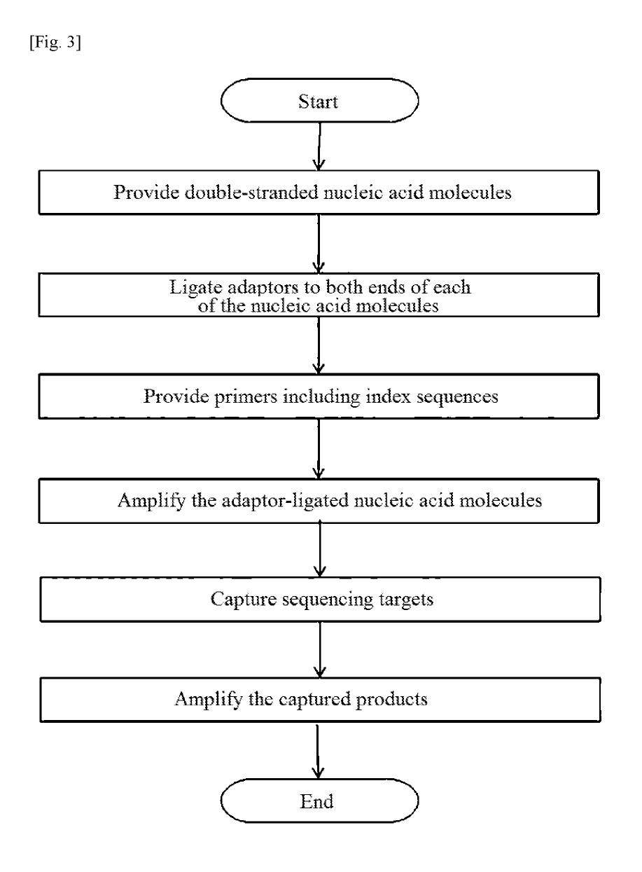

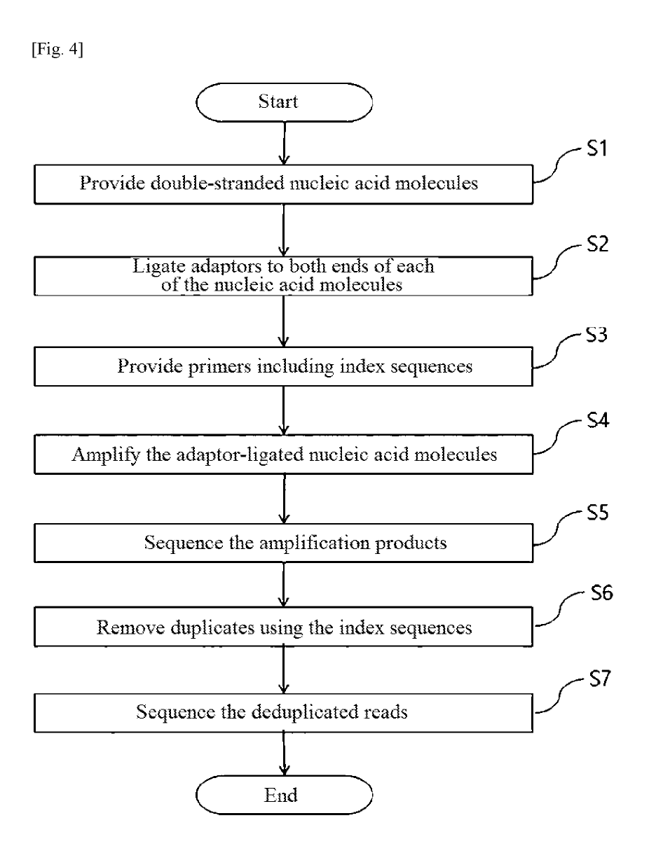

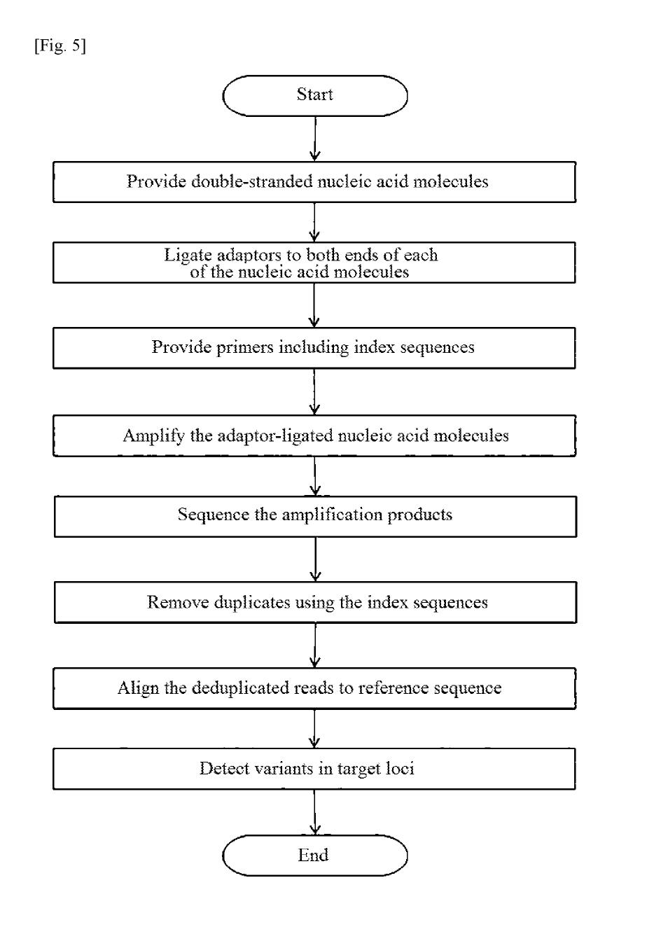

### MODE FOR CARRYING OUT THE INVENTION

The present invention will be explained in more detail with reference to the following examples. These examples are provided to assist in further understanding of the invention and are not intended to limit the spirit of the invention.

Cell-free DNA (cfDNA) can be extracted in only a very small amount. Further, since fragments of cell-free DNA (cfDNA) are wound around proteins in cell, many DNA molecules with similar structures are observed. For these reasons, a large proportion of PCR duplicates is detected in conventional assays, resulting in very low data efficiency. Thus, a method according to one embodiment of the present invention employs molecular barcoding of cfDNA and intends to identify an increase in sequencing depth by the molecular barcoding through data analysis.

### Example 1: Preparation of Libraries for Massively Parallel Sequencing

1.1. Ligation of Adaptor Sequences

cfDNA was extracted from plasma samples from three cancer patients using a cfDNA extraction kit (Qiagen) and libraries for sequencing the cfDNA were prepared by massively parallel sequencing by the following procedure. First, cfDNA fragments were replenished for their intact double-stranded forms. Then, one deoxyadenosine residue was appended on each of the 3′-ends of the nucleic acid molecules (dA-tailing) such that adaptors are ligated to both ends of each nucleic acid molecule in a predetermined direction. The adaptor molecules were ligated to the cfDNA fragments using a ligase (ligation). This procedure was carried out using a general library preparation kit for Illumina platform.

1.2. Introduction of Index Sequences

The cfDNA molecules terminated with the adaptor sequences were used as templates and PCR was performed to introduce index sequences. To this end, pairs of primers were used. Each primer pair consisted of a molecular index primer and a sample index primer. The molecular index primer included a sequence complementary to the corresponding adaptor, a sequence specific to the corresponding nucleic acid molecule, and a common primer sequence for sequencing. The sample index primer included a sample indexing sequence consisting of 8 nucleotides corresponding to index primers that are generally used to identify samples in Illumina platform. The common primer sequences located at both ends of the primer immobilize the DNA molecules onto a substrate of a sequencing device for sequencing through biochemical reactions. The molecular index primer and the sample index primer are set forth in SEQ ID NOS: 1 and 2, respectively:

5′-AATGATACGGCGACCACCGAGATCTACACNNNNNNNNACACTCTTTC
CCTACACGACGCTCTTCCGATC*T-3′ (1)

where 8 Ns shown in bold indicate the molecule-specific sequence and the asterisk (*) indicates a phosphorothioate bond,

5′-CAAGCAGAAGACGGCATACGAGATCGAGTAATGTGACTGGAGTTCAG
ACGTGTGCTCTTCCGATC*T-3′ (2)

where the underline indicates the sample indexing sequence and the asterisk (*) indicates a phosphorothioate bond.

PCR was performed using KAPA HiFi hotstart polymerase together with these primer sets to introduce the index sequences. Specifically, 50 μl of a PCR solution including 15 μl of the adaptor-ligated libraries, 5 μl of the molecular index primers, 5 μl of the sample index primers, and 25 μl of a KAPA library amplification mix was prepared. The solution was allowed to react at 98° C. for 45 sec. Thereafter, the reaction solution was subjected to 8-12 reaction cycles of 15 sec at 98° C., 30 sec at 65° C., and 1 min at 72° C. The final reaction was carried out at 72° C. for 10 min before storage at 4° C.

1.3. Capture of Target Nucleic Acids

Gene capture was performed by solution-based hybridization to analyze only tumor gene regions in the libraries into which the index sequences were incorporated. Solution-based hybridization is a technique in which DNA or RNA probes capable of complementary binding to capture target loci are constructed and mixed with DNA libraries on a solution to selectively capture nucleic acid molecules including the target loci. PCR amplification was performed to increase the total amount of the nucleic acid samples reduced after gene capture.

Specifically, the DNA library samples containing the index sequences incorporated thereinto were quantified and mixed with a blocking oligomer capable of complementary binding to the adaptor sequences to prevent the adaptors from being captured by similar sequences. The mixture was allowed to react at 95° C. for 5 min. The reaction products were mixed with a probe reagent for capturing the target loci and a hybridization buffer. The resulting hybridization solution was incubated at 65° C. for 16-24 h. Streptavidin T1 beads were washed with a washing buffer, mixed with the hybridization solution, and incubated at room temperature for 30 min. DNA captured on the beads was collected using a magnetic separator.

The captured DNA was amplified by PCR using the common primer sequences. 50 μl of a PCR solution including 15 μl of the captured DNA libraries, 2.5 μl of the forward primers, 2.5 μl of the reverse primers, and 25 μl of a KAPA library amplification mix was prepared. The solution was allowed to react at 98° C. for 45 sec. Thereafter, the reaction solution was subjected to 14-16 reaction cycles of 15 sec at 98° C., 30 sec at 65° C., and 1 min at 72° C. The final reaction was carried out at 72° C. for 10 min before storage at 4° C.

The amplified captured DNA libraries were purified using AMPure XP beads. The captured DNA library samples were found to have an average size of ˜300 bp, as analyzed using a TapeStation system.

### Example 2: Nucleic Acid Sequencing Through Massively Parallel Sequencing

The captured DNA library samples obtained in Example 1 were sequenced using a HiSeq2500 system (Illumina).

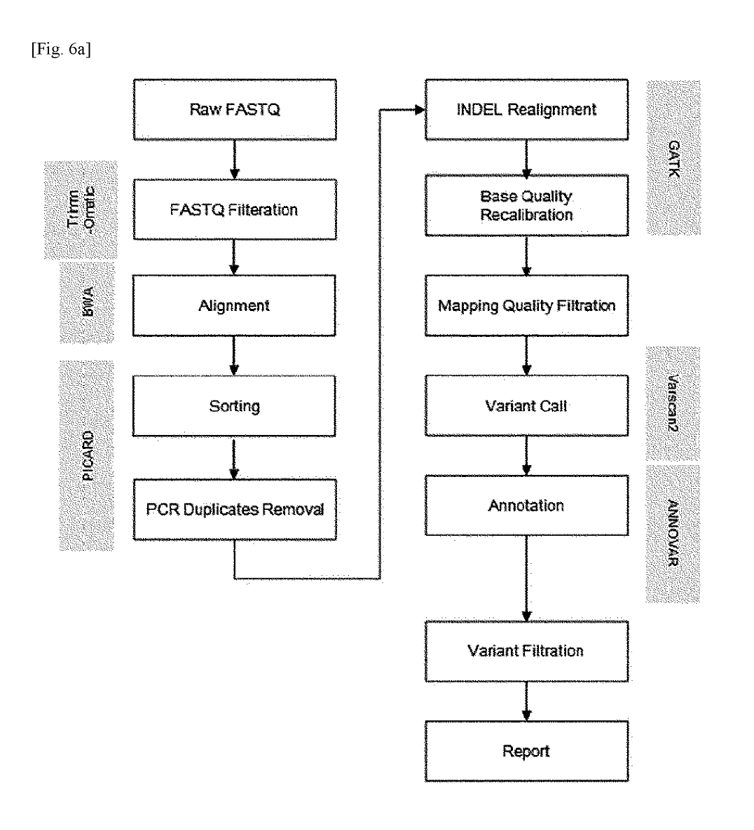

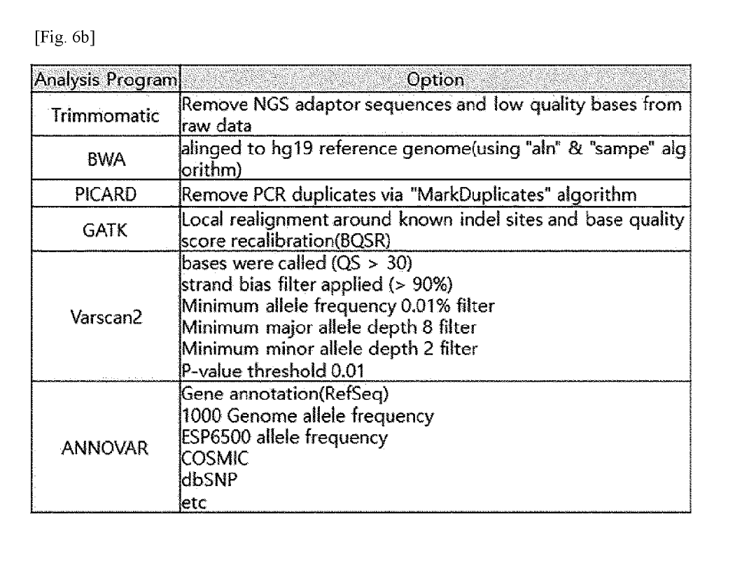

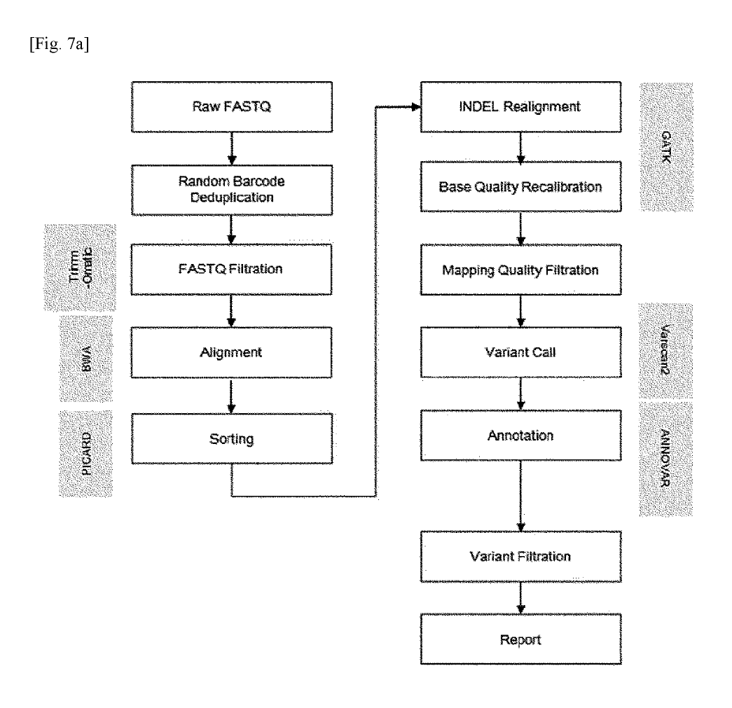

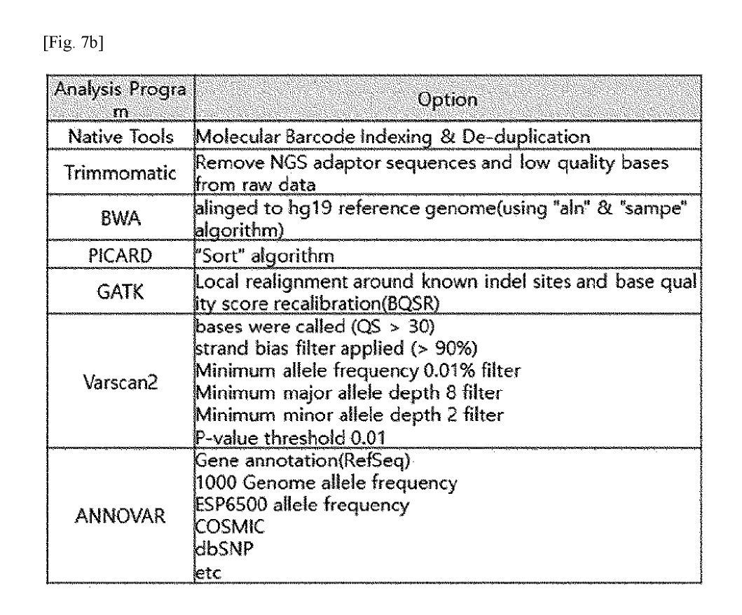

FIGS. 6a and 7a are flowcharts showing a general method for analyzing massively parallel sequencing data and a method for analyzing massively parallel sequencing data according to one embodiment, respectively. As shown in FIGS. 6a and 6b, the general data analysis method uses MarkDuplicates algorithm (Picard) for analyzing PCR duplicates based on the alignment locations of reads. In contrast, this experiment used an algorithm to previously perform deduplication using molecule-specific sequences at the initial stage of data analysis, as shown in FIGS. 7a and 7b.

Thereafter, the distributions of sequencing depths were plotted. The sequencing depths are values representing how many times the sequencing system reads the sequences of the target loci. The amount of data in the target loci obtained in this experiment was compared with that obtained in the conventional method.

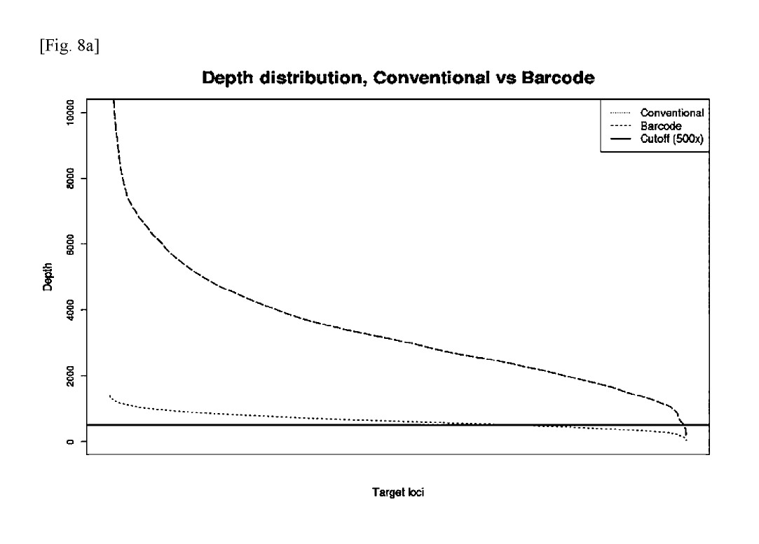

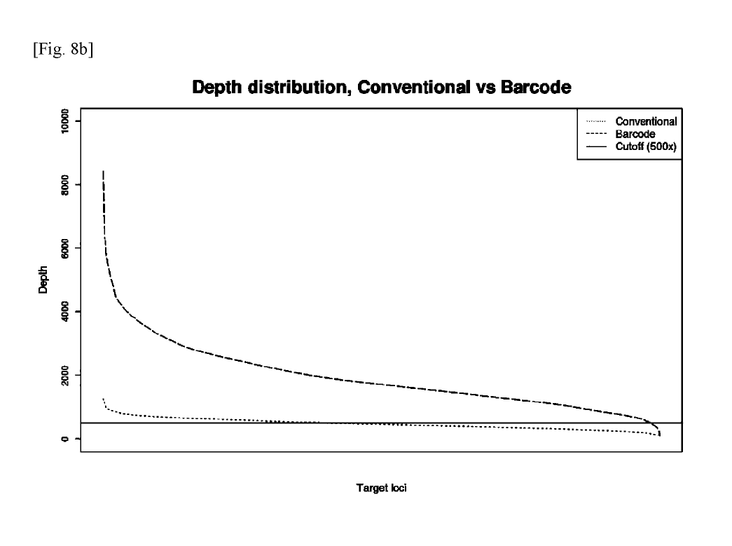

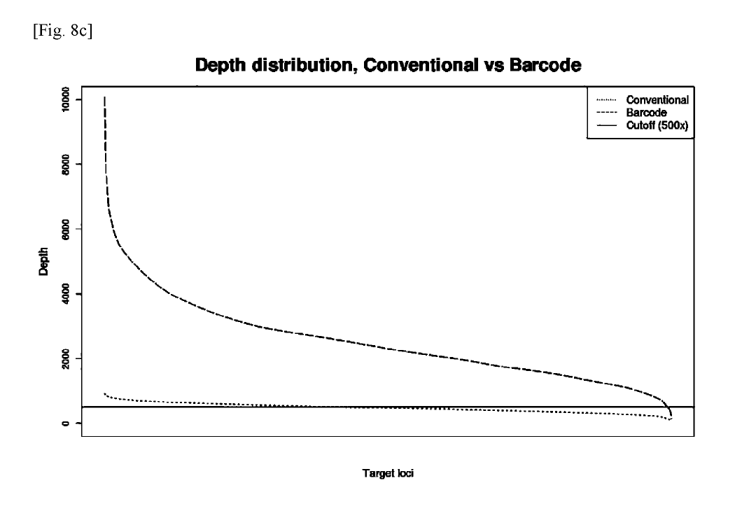

FIGS. 8a to 8c compare the results of analysis of sequencing data from three samples by the method according to one embodiment and the conventional method. In each curve, the pale grey line represents the distribution of the amounts of data obtained after the removal of duplicates based on the alignment locations of reads, as shown in FIG. 6, and the black line represents the distribution of the amounts of data obtained after the removal of duplicates using the molecule-specific sequences at the initial stage of analysis, as shown in FIG. 7. The red line represents the reference line of the amounts of data necessary for variant analysis.

As shown in FIGS. 8a to 8c, when the conventional method was used, a large proportion of data was eliminated by deduplication, resulting in overall low depth values. In contrast, when deduplication was previously performed using the molecule-specific sequences, overall high sequencing depth values were obtained, ensuring a broader area where the necessary amount of data for analysis can be acquired.

The amount of data in the target loci affects the detection sensitivity and accuracy of variant analysis. When the repeated reading of the corresponding location at least 500 times to detect a very small amount of variants (˜1%) while avoiding data errors is defined as a reference (500× cutoff), the target loci were found to be very broadly distributed below the reference value by the conventional analysis method whereas substantially all of the target loci were found to be distributed above the reference value by the method using the molecule-specific sequences.

1. A method for preparing libraries for massively parallel sequencing, comprising: providing two or more double-stranded nucleic acid molecules; ligating adaptors to both ends of each of the nucleic acid molecules; providing a pair of primers for amplifying each nucleic acid molecule wherein each of the paired primers comprises i) a 3′-end having a nucleotide sequence complementary to the corresponding adaptor, ii) a 5′-end having a common primer sequence for massively parallel sequencing, and iii) an index sequence located between the 3′-end and the 5′-end, one of the two index sequences is a sequence specific to the corresponding nucleic acid molecule, and the other index sequence is a sequence indexing a sample from which the nucleic acid molecule is derived; and performing amplification using the paired primers to obtain amplification products of the nucleic acid molecules comprising the molecule-specific sequences and the sample indexing sequences.

2. The method according to claim 1, wherein none of the adaptors comprise an index sequence.

3. The method according to claim 1, further comprising enzymatically cleaving the inner regions of the ligated adaptors.

4. The method according to claim 1, wherein each of the molecule-specific sequences consists of 4 to 20 nucleotides.

5. The method according to claim 1, wherein the number of the amplification cycles is 16 or less.

6. The method according to claim 1, further comprising capturing amplification products as sequencing targets.

7. The method according to claim 6, wherein the capture is performed by hybridization.

8. The method according to claim 6, further comprising amplifying the captured products using the common primer sequences.

9. A nucleic acid sequencing method through massively parallel sequencing, comprising: subjecting the libraries prepared by the method according claim 1 to massively parallel sequencing to obtain reads; removing duplicates having the same molecule-specific sequences and sample indexing sequences as the reads; and sequencing the deduplicated reads.

10. The method according to claim 9, wherein the massively parallel sequencing is selected from the group consisting of sequencing by synthesis, Ion Torrent sequencing, pyrosequencing, sequencing by ligation, nanopore sequencing, and single-molecule real-time sequencing.

11. The method according to claim 9, wherein the sequencing comprises aligning the deduplicated reads to a reference sequence.

12. The method according to claim 11, wherein some of the reads mapped to the same location during sequencing are not removed as duplicates.

13. The method according to claim 11, further comprising comparing the sequences of the aligned reads mapped to the target loci to detect sequence variants.

14. The method according to claim 13, wherein when the proportion of reads having the same sequence variants in the reads mapped to the target loci is below a predetermined value, the sequence variants are determined to be caused by sequencing errors.

15. A kit for preparing libraries for massively parallel sequencing, comprising a plurality of pairs of primers wherein each of the paired primers comprises a 3′-end having a nucleotide sequence complementary to adaptors ligated to both ends of a nucleic acid molecule, a 5′-end having a common primer sequence for massively parallel sequencing, and an index sequence located between the 3′-end and the 5′-end, one of the two index sequences is a sequence specific to the nucleic acid molecule, and the other index sequence is a sequence indexing a sample from which the nucleic acid molecule is derived.

16. The kit according to claim 15, wherein each of the molecule-specific sequences consists of 4 to 20 nucleotides.

17. The kit according to claim 15, wherein products obtained by amplification using the primers comprise the molecule-specific sequences and the sample indexing sequences in the flanking regions of the nucleic acid molecules.


SEQUENCE LISTING (2 sequences)

SEQ ID NO: 1 - DNA, 70 bp, Artificial Sequence
  Synthesized
  misc_feature (30)..(37): n is a, c, g, or t
aatgatacggcgaccaccgagatctacacnnnnnnnnacactctttccctacacgacgctcttccgatct

SEQ ID NO: 2 - DNA, 66 bp, Artificial Sequence
  Synthesized
caagcagaagacggcatacgagatcgagtaatgtgactggagttcagacgtgtgctcttccgatct